(19) United States
(12) Patent Application Publication
(10) Pub. No.: US 2008/0038329 A1
(43) Pub. Date: Feb. 14, 2008

(54) T-CELL ACTIVATING AGENT

(75) Inventors: Tetsuya Uchida, Saitama-shi (JP); Masahito Mori, Kawasaki-shi (JP); Hiroshi Oda, Kawasaki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: NOF CORPORATION, Tokyo (JP); The Director-General of National Institute of Infectious Diseases, Tokyo (JP)

(21) Appl. No.: 11/836,514

(22) Filed: Aug. 9, 2007

(30) Foreign Application Priority Data
Aug. 9, 2006 (JP) 2006-217270

(51) Int. Cl. A61K 9/127; A61K 39/00; A61P 37/00

(52) U.S. Cl. 424/450; 424/184.1; 424/277.1

(57) ABSTRACT

The present invention provides a T cell activator comprising an antigen-bound phospholipid membrane, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and wherein the antigen is bound to the surface of the phospholipid membrane.

### MUTUAL REFERENCE TO RELEVANT APPLICATION

This application is based on Japanese Patent Application No. 2006-217270 filed in Japan (filing date: Aug. 9, 2006), all teachings disclosed wherein are incorporated-herein by reference.

### TECHNICAL FIELD

The present invention relates to a T cell activator capable of efficiently and specifically enhancing cytotoxic T lymphocytes (CD8+ T cells, cytotoxic lymphocytes: CTL) for killing pathogen-infected cells or cancer cells, and useful in the prophylaxis/treatment for infectious disease or cancer.

More specifically, the present invention relates to a T cell activator comprising an antigen-bound phospholipid membrane, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and the antigen is bound to the surface of the phospholipid membrane.

### BACKGROUND ART

In animals such as humans, domestic animals, companion animals and the like, and fish, a variety of diseases due to infection of pathogens such as viruses to cells are known; and vaccines are widely used to prevent such infectious diseases.

To ameliorate or treat diseases caused by cell-infecting pathogens, such as HIV, mycoplasma, tuberculin, chlamydia, and malaria, it is necessary to effectively kill the cells infected with the pathogens.

This killing of pathogen-infected cells is possible by the action of cytotoxic T lymphocytes (hereinafter, CTLs), mainly CD8+ T cells; various investigations have been made on this method of effective enhancement of CTLs.

Although the effective enhancement of CTLs can be achieved by administering a live virus or an attenuated virus, this method cannot be a therapeutic method widely finding practical applications to have a sufficient effect in the medical sector because the subject is at a risk for being infected with the disease due to administration of the pathogen itself, and also because this method is only applicable to a limited kinds of pathogen, and for other reasons.

As a means for eliminating this risk for infection to the subject, many investigations have been made for identifying portions of pathogens, for example, proteins or peptides comprising antigen groups for triggering CTL attack responses for their specific cytotoxicity, preparing preparations of this antigenic substance alone or a combination of the antigenic substance and an adjuvant, and bringing them into practical application as vaccines for killing desired pathogen-infected cells. However, such a technology wherein portions of pathogens are used as major components of vaccines lacks a sufficient CTL enhancement effect, and no effective means for practical application has been obtained.

Regarding the aforementioned investigations, some investigations have been made for improving adjuvants to achieve desired effects. As adjuvants already in practical application or under investigation, aluminum hydroxide gel or oil-based adjuvants and the like can be mentioned, but none of them have achieved a sufficient CTL enhancement effect. When these adjuvants are used in combination, adverse reactions such as inflammation and allergies during ingestion of the adjuvant are often observed; in addition to the essential problem of CTL enhancement, investigations of adjuvants that are unlikely to cause adverse reactions are also required. This fact makes it more difficult to bring such vaccines into practical application.

In addition to the aforementioned diseases due to pathogen infection, conquering cancer is a major challenge in the modern medical sector. Amelioration or treatment for cancer by killing malignantly transformed cells, like the aforementioned amelioration/treatment for infectious disease, can be achieved by enhancing the CTL activity on cells that express tumor-specific antigens (tumor antigens) (malignantly transformed cells), and killing cancer cells.

As a means for ameliorating/treating cancer, an attempt has been made to ameliorate/treat cancer by combining a protein or peptide or the like, which is a tumor antigen, with aluminum hydroxide gel or an oil-based adjuvant, into a preparation, and administering this preparation as a cancer vaccine to the subject. However, no highly applicable, sufficiently effective technology for conquering various cancer diseases has been established.

Likewise, methods aiming to ameliorate/treat cancer but not cancer vaccination have been attempted. One of such methods is a method for performing cancer amelioration/treatment comprising collecting lymphocytes from the patient, activating the lymphocytes in vitro or enhancing the potential thereof for killing cancer cells, and then returning the lymphocytes to the patient's body. This method also lacks sufficient applicability and effect. Also, because this method follows the complex procedures of taking out the patient's lymphocytes from the body, treating them, and returning them into the body, this method involves a risk for adverse reactions due to this complicated procedure.

As stated above, there is a demand for a means for efficiently eliminating cells derived from self having become pathogenic, such as pathogen-infected cells and malignantly transformed cells in vivo; however, no sufficiently efficient and well practical technology as a pharmaceutical for clinical use has been found.

Meanwhile, various prior technologies for regulating the immune responses of the living body using a liposome preparation are known.

In patent documents 1 to 3 (JP 2005-145959, JP 9-12480 and JP 9-202735), liposomes wherein an antigen is bound to a phospholipid membrane are disclosed. In these documents, an effect of the liposomes on humoral immunity (effect to suppress IgE antibody production and increase IgG antibody production) is described but there is no description of action on cellular immunity (for example, CTL activating action).

In patent documents 4 (JP 3-236325), a liposome capable of killing virus-infected cells is disclosed. However, this liposome does not require an antigen in the liposome for expressing the cytotoxicity. No description is given regarding the CTL activating action of the liposome.

In patent document 5 (JP 2003-511421) and 6 (JP 2001-525668), liposomes comprising an antigen capable of activating T cells are disclosed. However, in these liposomes, the antigen is encapsulated in the liposomes and not bound onto the liposomes.

In patent document 7 (JP 2002-526436), an immunogenic liposome composition comprising a vesicle-forming lipid and an antigen construct comprising one or more antigen determinants and a hydrophobic domain, wherein the hydrophobic domain is bound to a membrane of the aforementioned liposome composition, is disclosed. In Examples, as phospholipids that constitute particularly preferable liposomes, dimyristoylphosphatidylcholine, dimyristoylphosphatidylglycerol and the like are used.

In patent document 8 (EP 0203676), a vaccine for generating immunogenic T cell response protective against virus, said vaccine comprising 1) a peptide-fatty acid conjugate, 2) a liposome composition comprising a mixture of phosphatidylcholine, cholesterol and lisophosphatidylcholine, and 3) an adjuvant, is disclosed. In no Examples, the choice (carbon number) of the phospholipid (phosphatidylcholine) used to prepare the liposome is specified.

Although as the method for eliminating pathogenic cells, enhancement of the cellular immune potential (CTL, CD8+ T cells) essentially possessed by the living body is desirable, no liposome preparations capable of enhancing the cellular immune potential to a fully satisfactory level are known.

### DISCLOSURE OF THE INVENTION

It is a problem to be solved by the present invention to provide a T cell activator capable of efficiently and specifically enhancing cytotoxic T lymphocytes (CD8+ T cells, cytotoxic lymphocytes: CTLs) for killing pathogen-infected cells or cancer cells, and useful for the prophylaxis/treatment for infectious disease and cancer.

The present inventors diligently investigated to solve the above-described problem and, as a result, found that using a phospholipid membrane comprising a phospholipid having an acyl group having one unsaturated bond and 14 to 24 carbon atoms or a hydrocarbon group having one unsaturated bond and 14 to 24 carbon atoms, and a phospholipid membrane stabilizer, wherein an antigen is bound to the surface thereof, cytotoxic T lymphocytes can be efficiently and specifically enhanced, and developed the present invention. Accordingly, the present invention provides the following:
[1] A T cell activator comprising an antigen-bound phospholipid membrane, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and the antigen is bound to the surface of the phospholipid membrane.
   [2] The T cell activator described in [1] above, wherein the phospholipid is a phospholipid having an acyl group having one unsaturated bond and 14 to 24 carbon atoms.
   [3] The T cell activator described in [2] above, wherein the acyl group is an oleoyl group.
   [4] The T cell activator described in [2] above, wherein the phospholipid is at least one selected from among diacylphosphatidylserine, diacylphosphatidylglycerol, diacylphosphatidic acid, diacylphosphatidylcholine, diacylphosphatidylethanolamine, succinimidyl-diacylphosphatidylethanolamine, and maleimide-diacylphosphatidylethanolamine.
   [5] The T cell activator described in [1] above, wherein the phospholipid membrane stabilizer is cholesterol.
   [6] The T cell activator described in [1] above, wherein the antigen is bound to the phospholipid having an acyl group having one unsaturated bond and 14 to 24 carbon atoms or a hydrocarbon group having one unsaturated bond and 14 to 24 carbon atoms, contained in the phospholipid membrane.
   [7] The T cell activator described in [1] above, wherein the antigen is an antigen derived from an intracellular infectious pathogen or a tumor antigen.
   [8] The T cell activator described in [1] above, wherein the phospholipid membrane is a liposome.
   [9] The T cell activator described in [1] above, wherein the T cells are CD8+ T cells.
   [10] The T cell activator described in [1] above, wherein the T cells are CTLs.
   [11] The T cell activator described in [1] above, wherein the phospholipid membrane has the following composition:

(A) a phospholipid having an acyl group having one unsaturated bond and 14 to 24 carbon atoms or a hydrocarbon group having one unsaturated bond and 14 to 24 carbon atoms, 1 to 99.8 mol %;

(B) a phospholipid membrane stabilizer, 0.2 to 75 mol %.

[12] A T cell activator comprising an antigen-bound phospholipid membrane having the following composition:

(I) an acidic phospholipid having an acyl group having one unsaturated bond and 14 to 24 carbon atoms or a hydrocarbon group having one unsaturated bond and 14 to 24 carbon atoms, 1 to 85 mol %;

(II) a neutral phospholipid having an acyl group having one unsaturated bond and 14 to 24 carbon atoms or a hydrocarbon group having one unsaturated bond and 14 to 24 carbon atoms, 0.01 to 80 mol %;

(III) an antigen-bound phospholipid having an acyl group having one unsaturated bond and 14 to 24 carbon atoms or a hydrocarbon group having one unsaturated bond and 14 to 24 carbon atoms, 0.2 to 80 mol %;

(IV) a phospholipid membrane stabilizer, 0.2 to 75 mol %.

[13] A phospholipid membrane for use as a T cell activator, comprising a phospholipid having an acyl group having one unsaturated bond and 14 to 24 carbon atoms or a hydrocarbon group having one unsaturated bond and 14 to 24 carbon atoms, and a phospholipid membrane stabilizer, wherein an antigen is bound to the surface thereof.

[14] A method of activating T cells in a mammal, comprising administering an effective amount of an antigen-bound phospholipid membrane to the mammal, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and the antigen is bound to the surface of the phospholipid membrane.

[15] The method described in [14] above, wherein the phospholipid is a phospholipid having an acyl group having one unsaturated bond and 14 to 24 carbon atoms.

[16] The method described in [15] above, wherein the acyl group is an oleoyl group.

[17] The method described in [15] above, wherein the phospholipid is at least one selected from among diacylphosphatidylserine, diacylphosphatidylglycerol, diacylphosphatidic acid, diacylphosphatidylcholine, diacylphosphatidylethanolamine, succinimidyl-diacylphosphatidylethanolamine, and maleimide-diacylphosphatidylethanolamine.

[18] The method described in [14] above, wherein the phospholipid membrane stabilizer is cholesterol.

[19] The method described in [14] above, wherein the antigen is bound to the phospholipid having an acyl group having one unsaturated bond and 14 to 24 carbon atoms or a hydrocarbon group having one unsaturated bond and 14 to 24 carbon atoms, contained in the phospholipid membrane.

[20] The method described in [14] above, wherein the antigen is an antigen derived from an intracellular infectious pathogen or a tumor antigen.

[21] The method described in [14] above, wherein the phospholipid membrane is a liposome.

[22] The method described in [14] above, wherein the T cells are CD8+ T cells.

[23] The method described in [14] above, wherein the T cells are CTLs.

[24] The method described in [14] above, wherein the phospholipid membrane has the following composition:

(A) a phospholipid having an acyl group having one unsaturated bond and 14 to 24 carbon atoms or a hydrocarbon group having one unsaturated bond and 14 to 24 carbon atoms, 1 to 99.8 mol %;

(B) a phospholipid membrane stabilizer, 0.2 to 75 mol %.

[25] A method of activating T cells in a mammal, comprising administering an effective amount of an antigen-bound phospholipid membrane having the following composition to the mammal:

(I) an acidic phospholipid having an acyl group having one unsaturated bond and 14 to 24 carbon atoms or a hydrocarbon group having one unsaturated bond and 14 to 24 carbon atoms, 1 to 85 mol %;

(II) a neutral phospholipid having an acyl group having one unsaturated bond and 14 to 24 carbon atoms or a hydrocarbon group having one unsaturated bond and 14 to 24 carbon atoms, 0.01 to 80 mol %;

(III) an antigen-bound phospholipid having an acyl group having one unsaturated bond and 14 to 24 carbon atoms or a hydrocarbon group having one unsaturated bond and 14 to 24 carbon atoms, 0.2 to 80 mol %;

(IV) a phospholipid membrane stabilizer, 0.2 to 75 mol %.

### EFFECT OF THE INVENTION

Using the T cell activator of the present invention, cytotoxic T lymphocytes (CD8+ T cells, CTL) for killing pathogen-infected cells or cancer cells can be efficiently and specifically enhanced to prevent/treat infectious disease or cancer.

### BEST MODE FOR CARRYING OUT THE INVENTION

The present invention provides a T cell activator comprising an antigen-bound phospholipid membrane, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and the antigen is bound to the surface of the phospholipid membrane.

The phospholipid membrane used in the T cell activator of the present invention has a structure wherein a phospholipid, which is an amphiphilic surfactant, forms an interface with the polar group facing the water phase side and the hydrophobic group facing the opposite side of the interface. As examples of the phospholipid membrane structure, a liposome, a phospholipid bilayer membrane, a phospholipid miselle, a phospholipid emulsion and the like can be mentioned. Here, a liposome refers to a phospholipid bilayer membrane having a closed space. The phospholipid miselle and the phospholipid emulsion have a phospholipid monolayer membrane structure.

Considering practical applicability, ease of preparation design, convenience for manufacture and quality control, and the like, the phospholipid membrane is preferably a liposome or a phospholipid miselle, most preferably a liposome.

The antigen used in the T cell activator of the present invention is not subject to limitation, as long as T cells (for example, cytotoxic T lymphocytes) can recognize it as an antigen; all substances that can become antigens in humans; companion animals such as dogs, cats and small birds; and domestic animals such as chicken, ducks, pigs, bovine, and sheep can be used. As the antigen, specifically, for example, an antigen derived from an intracellular infectious pathogen, an antigen associated with malignantly transformed cells (tumor antigens) and the like can be used. These antigens are known to be recognized by cytotoxic T lymphocytes.

As the antigen derived from an intracellular infectious pathogen, a pathogen per se or a portion thereof, or an inactivated or attenuated pathogen per se or a portion thereof and the like can be mentioned. As examples of these antigens, various toxoids such as those of tetanus and diphtheria; virus-derived antigens such as those of influenza, poliomyelitis, Japanese encephalitis, measles, mumps, rubella, rabies, yellow fever, varicella, hepatitis A, hepatitis B, hepatitis C, hemorrhagic fever with renal syndrome, Dengue hemorrhagic fever, rotavirus infectious disease, parvovirus, corona virus, distemper virus, leptospira, infectious bronchitis virus, contagious leukemia virus, and AIDS; antigens derived from bacteria such as mycoplasmas; antigens derived from intracellular parasitic protozoas such as plasmodium and schistosomes and the like can be mentioned. The above-described antigens can be used alone or in combination of two or more kinds.

The antigen associated with malignantly transformed cells is not subject to limitation, as long as it is a protein, carbohydrate chain, peptide or the like specifically expressed in malignantly transformed cells; for example, histocompatibility antigens specific for breast cancer, gastric cancer, liver cancer, lung cancer and the like, tumor-specific transplantation antigens (TSTA), tumor associated antigens (TAA) and the like can be mentioned. Specifically, as the antigen associated with malignantly transformed cells, a-fetoprotein (a-FP), PIVKA-2, CEA, CA19-9, CA125, CA15-3, CYFRA, nerve-specific enolase (NSE), prostate-specific antigen (PSA), a-fetoprotein L3 fraction (AFP-L3), total a-fetoprotein (Total-AFP), NCC-ST439, CEA in nipple secretions (LANA MAMMO CEA), SCC and the like can be mentioned. The above-described antigens can be used alone or in combination of two or more kinds.

Antigens are proteins, peptides or saccharides and the like, and are capable of binding to the surface of a phospholipid membrane via a functional group possessed by the antigen. As the functional group in the antigen, used for binding to the phospholipid membrane surface, an amino group, a thiol group, a carboxyl group, a hydroxyl group, a disulfide group or a hydrophobic group consisting of a hydrocarbon group having a methylene chain and the like can be mentioned. These functional groups are capable of binding the antigen to the phospholipid membrane surface via a covalent bond for an amino group, a thiol group, a carboxyl group, a hydroxyl group and a disulfide group, via an ionic bond for an amino group and a carboxyl group, and via a hydrophobic bond for hydrophobic groups. Because antigens are often proteins or peptides, the functional group content ratio is high and practical application is easy. From this viewpoint, the antigen preferably binds to the phospholipid membrane surface via an amino group, a carboxyl group or a thiol group. When the antigen is a saccharide, it is preferable, from the same viewpoint, that the antigen bind to the phospholipid membrane surface via a hydroxyl group.

Because the antigen binds stably to the phospholipid membrane via a functional group possessed by the antigen, the phospholipid membrane desirably has a functional group such as an amino group, a succinimide group, a maleimide group, a thiol group, a carboxyl group, a hydroxyl group, a disulfide group, or a hydrophobic group consisting of a hydrocarbon group having a methylene chain. When the antigen is a protein or a peptide, the functional group possessed by the corresponding phospholipid membrane is preferably an amino group, a succinimide group or a maleimide group. A combination of a functional group possessed by the antigen and a functional group possessed by the phospholipid membrane, involved in the binding of the antigen to the phospholipid membrane, can be optionally chosen, as long as the effect of the present invention is not influenced; as preferable combinations, a combination of an amino group and an aldehyde group, a combination of an amino group and an amino group, a combination of an amino group and a succinimide group, a combination of a thiol group and a maleimide group and the like can be mentioned. Ionic bonds and hydrophobic bonds are preferable because of convenience in the procedure of binding the antigen to the phospholipid membrane, and the ease of preparation of the T cell activator; covalent bonds are preferable because of the stability of the binding of the antigen to the phospholipid membrane surface or because of storage stability during actual use of the T cell activator. A feature of the T cell activator of the present invention resides in that an antigen is bound to the surface of a phospholipid membrane which is a constituent of the activator, whereby an excellent T cell (cytotoxic T lymphocyte) activating effect is achieved. Therefore, it is preferable, because of further enhancement of the effect of the present invention, that the antigen remains stably bound to the phospholipid membrane surface even after the antigen is administered to a living organism by, for example, injection, during the actual use. From this viewpoint, the bond between the antigen and the phospholipid membrane is preferably a covalent bond.

The antigen-bound phospholipid membrane used in the T cell activator of the present invention comprises a phospholipid having an acyl group having one unsaturated bond and 14 to 24 carbon atoms or a hydrocarbon group having one unsaturated bond and 14 to 24 carbon atoms, and a phospholipid membrane stabilizer.

In the phospholipid having an acyl group having one unsaturated bond and 14 to 24 carbon atoms, the carbon number for the acyl group is preferably 16 to 22, more preferably 18 to 22, most preferably 18. As the acyl group, specifically, a palmitoleoyl group, an oleoyl group, an erucoyl group and the like can be mentioned, and the acyl group is most preferably an oleoyl group.

In the phospholipid having a hydrocarbon group having one unsaturated bond and 14 to 24 carbon atoms, the carbon number for the hydrocarbon group is preferably 16 to 22, more preferably 18 to 22, most preferably 18. As the hydrocarbon group, specifically, a tetradecenyl group, a hexadecenyl group, an octadecenyl group, a C20 monoen group, a C22 monoen group, a C24 monoen group and the like can be mentioned.

The unsaturated acyl groups or unsaturated hydrocarbon groups bound to the 1-position and the 2-position of the glycerin residue possessed by the phospholipid may be identical or different. From the viewpoint of industrial productivity, it is preferable that the groups at the 1-position and the 2-position be identical.

As the phospholipid, a phospholipid having an acyl group having one unsaturated bond and 14 to 24 carbon atoms is preferably used.

An object of the present invention is to efficiently and specifically enhance cytotoxic T lymphocytes (CD8+ T cells, CTL) for killing pathogen-infected cells or cancer cells. To enhance CTL activity to a practically sufficient level, the phospholipid preferably has an acyl group having one unsaturated bond and 14 to 24 carbon atoms. If the carbon number for the acyl group is less than 13, the stability of the liposome worsens or the CTL activity enhancing effect is insufficient in some cases. If the carbon number for the acyl group exceeds 24, the stability of the liposome worsens in some cases.

As the phospholipid having an acyl group having one unsaturated bond and 14 to 24 carbon atoms or a hydrocarbon group having one unsaturated bond and 14 to 24 carbon atoms, an acidic phospholipid, a neutral phospholipid, a reactive phospholipid having a functional group capable of binding an antigen and the like can be mentioned. It is possible to choose kinds or ratios of these phospholipids as appropriate according to various requirements.

As the acidic phospholipid, phosphatidylserine, phosphatidylglycerol, phosphatidic acid, phosphatidylinositol and the like can be used. Considering enhancement of CTL activity to a practically sufficient level, and industrial suppliability, quality for use as a pharmaceutical and the like, diacylphosphatidylserine, diacylphosphatidylglycerol, diacylphosphatidic acid, and diacylphosphatidylinositol, having an acyl group having one unsaturated bond and 14 to 24 carbon atoms, are preferably used. The acidic phospholipid confers an anionic ionized group to the phospholipid membrane surface, thus providing a minus zeta potential to the phospholipid membrane surface. For this reason, the phospholipid membrane acquires a repulsive force in charge and can present as a stable preparation in aqueous solvents. Hence, an acidic phospholipid is important in assuring the stability of the phospholipid membrane when the T cell activator is present in an aqueous solvent.

As examples of the neutral phospholipid, phosphatidylcholine and the like can be used. It is possible to choose kinds or quantities of the neutral phospholipids that can be used in the present invention as appropriate, as long as CTL activity enhancement, an object of the present invention, is accomplished. Neutral phospholipids, compared to acidic phospholipids and antigen-bound phospholipids, are more highly functional to stabilize the phospholipid membrane, and are capable of improving the membrane stability. From this viewpoint, the phospholipid membrane contained in the T cell activator of the present invention preferably comprises a neutral phospholipid. While assuring sufficient contents of the acidic phospholipids, the reactive phospholipid for antigen binding and the phospholipid membrane stabilizer, used to accomplish a CTL activity enhancing effect, the amount of neutral phospholipid used can be determined.

In the T cell activator of the present invention, the antigen binds to the phospholipid membrane surface by binding to the phospholipid having an acyl group having one unsaturated bond and 14 to 24 carbon atoms or the hydrocarbon group having one unsaturated bond and 14 to 24 carbon atoms, contained in the phospholipid membrane.

As the phospholipid for antigen binding, a reactive phospholipid having a functional group to which the antibody can bind is used. It is possible to choose kinds or ratios of the reactive phospholipid having an acyl group having one unsaturated bond and 14 to 24 carbon atoms or a hydrocarbon group having one unsaturated bond and 14 to 24 carbon atoms as appropriate according to various requirements. In the reactive phospholipid, like in the aforementioned phospholipid, it is undesirable that the carbon number for the unsaturated acyl group or unsaturated hydrocarbon group contained in the phospholipid exceeds 24 or is less than 14.

As the reactive phospholipid, phosphatidylethanolamine or a terminally modified product thereof can be mentioned. Phosphatidylglycerol, phosphatidylserine, phosphatidic acid, phosphatidylinositol and terminally modified products thereof can also be used as the reactive phospholipid. Considering industrial availability, convenience of the step for binding to the antigen, yield and the like, phosphatidylethanolamine or a terminally modified product thereof is preferably used. Phosphatidylethanolamine has an amino group capable of being bound by an antibody at a terminus thereof. Furthermore, considering enhancement of CTL activity to a practically sufficient level, stability in the phospholipid membrane, industrial suppliability, quality for use as a pharmaceutical and the like, a diacylphosphatidylethanolamine having an acyl group having one unsaturated bond and 14 to 24 carbon atoms or a terminally modified product thereof is most preferably used.

Diacylphosphatidylethanolamine can be obtained from diacylphosphatidylcholine as the starting material by, for example, allowing choline and ethanolamine to undergo a base exchange reaction using phospholipase D. Specifically, a solution of diacylphosphatidylcholine in chloroform and solution of phospholipase D and ethanolamine in water are mixed at an appropriate ratio to yield a crude reaction product. The crude reaction product may be purified by silica gel column using a chloroform/methanol/aqueous solvent to yield the object diacylphosphatidylethanolamine. Those skilled in the art are able to choose column purification conditions such as solvent composition ratio as appropriate and perform this purification.

As the terminally modified product, a terminally modified diacylphosphatidylethanolamine prepared by binding one terminal of a bifunctional reactive compound to the amino group of diacylphosphatidylethanolamine can be mentioned. As the bifunctional reactive compound, a compound having an aldehyde group or succinimide group capable of reacting with the amino group of diacylphosphatidylethanolamine at least one terminal thereof can be utilized. As the bifunctional reactive compound having an aldehyde group, glyoxal, glutaraldehyde, succinedialdehyde, terephthalaldehyde and the like can be mentioned. Preferably, glutaraldehyde can be mentioned. As the bifunctional reactive compound having a succinimide group, dithiobis(succinimidylpropionate), ethylene glycol-bis (succinimidylsuccinate), disuccinimidylsuccinate, disuccinimidylsuberate, disuccinimidylglutarate and the like can be mentioned.

As the bifunctional reactive compound having a succinimide group on one terminal and a maleimide group on the other terminal, N-succinimidyl-4-(p-maleimidephenyl)butyrate, sulfosuccinimidyl-4-(p-maleimidephenyl)butyrate, N-succinimidyl-4-(p-maleimidephenyl)acetate, N-succinimidyl-4-(p-maleimidephenyl)propionate, succinimidyl-4-(N-maleimideethyl)-cyclohexane-1-carboxylate, sulfosuccinimidyl-4-(N-maleimideethyl)-cyclohexane-1-carboxylate, N-(γ-maleimidebutyryloxy)succinimide, N-(ε-maleimidecaproyloxy)succinimide and the like can be mentioned. Using these bifunctional reactive compound, a terminally modified diacylphosphatidylethanolamine having a maleimide group as the functional group is obtained. The functional group at one terminal of such a bifunctional reactive compound can be bound to the amino group of diacylphosphatidylethanolamine to obtain a terminally modified diacylphosphatidylethanolamine.

As an example of the method of binding an antigen to the phospholipid membrane surface, a method can be mentioned wherein a phospholipid membrane comprising the above-described reactive phospholipid is prepared, and then an antigen is added to bind the antigen to the reactive phospholipid in the phospholipid membrane. Also, by binding an antigen to a reactive phospholipid in advance, and then mixing the resulting antigen-bound reactive phospholipid with a phospholipid other than the reactive phospholipid and a phospholipid membrane stabilizer, a phospholipid membrane wherein the antigen is bound to the surface thereof can also be obtained. The method of binding an antigen to a reactive phospholipid is well known in the art.

The phospholipid membrane contained in the T cell activator of the present invention comprises at least one kind, for example, two kinds or more, preferably three kinds or more, of a phospholipid having an acyl group having one unsaturated bond and 14 to 24 carbon atoms or a hydrocarbon group having one unsaturated bond and 14 to 24 carbon atoms. For example, the phospholipid membrane contained in the T cell activator of the present invention comprises at least one kind, for example, two kinds or more, preferably three kinds or more, of a phospholipid having an acyl group having one unsaturated bond and 14 to 24 carbon atoms or a hydrocarbon group having one unsaturated bond and 14 to 24 carbon atoms, selected from among diacylphosphatidylserine, diacylphosphatidylglycerol, diacylphosphatidic acid, diacylphosphatidylcholine, diacylphosphatidylethanolamine, succinimidyl-diacylphosphatidylethanolamine, and maleimide-diacylphosphatidylethanolamine.

The phospholipid membrane contained in the T cell activator of the present invention preferably comprises at least one kind of each of:

an acidic phospholipid having an acyl group having one unsaturated bond and 14 to 24 carbon atoms or a hydrocarbon group having one unsaturated bond and 14 to 24 carbon atoms, a neutral phospholipid having an acyl group having one unsaturated bond and 14 to 24 carbon atoms or a hydrocarbon group having one unsaturated bond and 14 to 24 carbon atoms, and a reactive phospholipid having an acyl group having one unsaturated bond and 14 to 24 carbon atoms or a hydrocarbon group having one unsaturated bond and 14 to 24 carbon atoms.

In the present invention, as the phospholipid membrane stabilizer, sterols or tocopherols can be used. As the sterols, those generally known as sterols may be used, such as, cholesterol, sitosterol, campesterol, stigmasterol, brassicasterol and the like. Considering availability and the like, cholesterol is particularly preferably used. As the tocopherols, those generally known as a tocopherol may be used; for example, considering availability and the like, commercially available α-tocopherol is preferably mentioned.

Furthermore, the antigen-bound phospholipid membrane contained in the T cell activator of the present invention may comprise a commonly known phospholipid membrane constituent capable of constituting a phospholipid membrane, as long as the effect of the present invention is not affected.

As examples of the composition of the antigen-bound phospholipid membrane contained in the T cell activator of the present invention, the following can be mentioned:

(A) a phospholipid having an acyl group having one unsaturated bond and 14 to 24 carbon atoms or a hydrocarbon group having one unsaturated bond and 14 to 24 carbon atoms, 1 to 99.8 mol %;

(B) a phospholipid membrane stabilizer, 0.2 to 75 mol %.

The content of each component is expressed as mol % relative to all constituents of the antigen-bound phospholipid membrane.

The content of the above-described component (A), from the viewpoint of phospholipid membrane stability, is preferably 10 to 90 mol %, more preferably 30 to 80 mol %, still more preferably 50 to 70 mol %.

The content of the above-described component (B), from the viewpoint of phospholipid membrane stability, is preferably 5 to 70 mol %, more preferably 10 to 60 mol %, still more preferably 20 to 50 mol %. If the stabilizer content exceeds 75 mol %, the phospholipid membrane stability is affected and this is undesirable.

The above-described component (A) comprises the following:

(a) an antigen-unbound phospholipid having an acyl group having one unsaturated bond and 14 to 24 carbon atoms or a hydrocarbon group having one unsaturated bond and 14 to 24 carbon atoms, and

(b) an antigen-bound phospholipid having an acyl group having one unsaturated bond and 14 to 24 carbon atoms or a hydrocarbon group having one unsaturated bond and 14 to 24 carbon atoms.

The content of the above-described component (a) is generally 0.01 to 85 mol %, preferably 0.1 to 80 mol %, more preferably 0.1 to 60 mol %, still more preferably 0.1 to 50 mol %.

The content of the above-described component (b) is generally 0.2 to 80 mol %, preferably 0.3 to 60 mol %, more preferably 0.4 to 50 mol %, still more preferably 0.5 to 25 mol %. If the content is less than 0.2 mol %, the amount of antigen decreases, and therefore it becomes difficult to activate cytotoxic T lymphocytes to a practically sufficient level; if the content exceeds 80 mol %, the phospholipid membrane stability will decrease.

The phospholipid of the above-described component (a) generally includes the above-described acidic phospholipid and neutral phospholipid. The phospholipid of the above-described component (b) includes the above-described reactive phospholipid.

The content of the acidic phospholipid is generally 1 to 85 mol %, preferably 2 to 80 mol %, more preferably 4 to 60 mol %, still more preferably 5 to 40 mol %. If the content is less than 1 mol %, the zeta potential lowers, the phospholipid membrane stability decreases, and it becomes difficult to activate cytotoxic T lymphocytes to a practically sufficient level. If the content exceeds 85 mol %, the content of antigen-bound phospholipid in the phospholipid membrane decreases, and it becomes difficult to activate cytotoxic T lymphocytes to a practically sufficient level.

The content of the neutral phospholipid is generally 0.01 to 80 mol %, preferably 0.1 to 70 mol %, more preferably 0.1 to 60 mol %, still more preferably 0.1 to 50 mol %. If the content exceeds 80.0 mol %, the contents of the acidic phospholipid, antigen-bound phospholipid and phospholipid membrane stabilizer contained in the phospholipid membrane decrease, and it becomes difficult to activate cytotoxic T lymphocytes to a practically sufficient level.

The antigen-bound phospholipid is obtained by binding the antigen to the aforementioned reactive phospholipid; the ratio of the reactive phospholipid which binds to the antigen can be chosen by appropriately setting the kind of functional group used for the binding, binding treatment conditions and the like, as long as the effect of the present invention is not interfered with.

For example, when a terminally modified diacylphosphatidylethanolamine obtained by binding one terminal of disuccinimidylsuccinate, which is a bifunctional reactive compound, to the terminal amino group of diacylphosphatidylethanolamine, is used as the reactive phospholipid, 10 to 99% of the reactive phospholipids can be bound to the antigen by choosing appropriate binding treatment conditions. In this case, the reactive phospholipids unbound to the antigen become acidic phospholipids and come to be contained in the phospholipid membrane.

As a preferable embodiment of the antigen-bound phospholipid membrane contained in the T cell activator of the present invention, the following composition can be mentioned:

(I) an acidic phospholipid having an acyl group having one unsaturated bond and 14 to 24 carbon atoms or a hydrocarbon group having one unsaturated bond and 14 to 24 carbon atoms, 1 to 85 mol %;

(II) a neutral phospholipid having an acyl group having one unsaturated bond and 14 to 24 carbon atoms or a hydrocarbon group having one unsaturated bond and 14 to 24 carbon atoms, 0.01 to 80 mol %;

(III) an antigen-bound phospholipid having an acyl group having one unsaturated bond and 14 to 24 carbon atoms or a hydrocarbon group having one unsaturated bond and 14 to 24 carbon atoms, 0.2 to 80 mol %;

(IV) a phospholipid membrane stabilizer, 0.2 to 75 mol %.

(Total 100 mol %)

As a more preferable embodiment of the antigen-bound phospholipid membrane contained in the T cell activator of the present invention, the following composition can be mentioned:

Above-described component (I), 2 to 80 mol %

Above-described component (II), 0.1 to 70 mol %

Above-described component (III), 0.3 to 60 mol %

Above-described component (IV), 10 to 70 mol %

(Total 100 mol %)

As a still more preferable embodiment of the antigen-bound phospholipid membrane contained in the T cell activator of the present invention, the following composition can be mentioned:

Above-described component (I), 4 to 60 mol %

Above-described component (II), 0.1 to 60 mol %

Above-described component (III), 0.4 to 50 mol %

Above-described component (IV), 20 to 60 mol %

(Total 100 mol %)

As a particularly preferable embodiment of the antigen-bound phospholipid membrane contained in the T cell activator of the present invention, the following composition can be mentioned:

Above-described component (I), 5 to 40 mol %

Above-described component (II), 0.1 to 50 mol %

Above-described component (III), 0.5 to 25 mol %

Above-described component (IV), 25 to 55 mol %

(Total 100 mol %)

Although the T cell activator of the present invention is characterized in that the carbon number for the unsaturated acyl group or unsaturated hydrocarbon group contained in the phospholipid in the phospholipid membrane contained in the activator is 14 to 24, the activator may comprise a phospholipid comprising an unsaturated acyl group or unsaturated hydrocarbon group having less than 14 or exceeding 24 carbon atoms, as long as the effect of the present invention is not interfered with. The ratio by number of the unsaturated acyl groups or unsaturated hydrocarbon groups having 14 to 24 carbon atoms relative to the total number of all unsaturated acyl groups or unsaturated hydrocarbon groups contained in the phospholipid in the phospholipid membrane contained in the T cell activator of the present invention is, for example, not less than 50%, preferably not less than 60%, more preferably not less than 75%, still more preferably not less than 90%, most preferably not less than 97% (for example, substantially 100%).

The phospholipid membrane contained in the T cell activator of the present invention may comprise another lipid, besides phospholipids, having an acyl group or hydrocarbon group whose carbon number is in the range of 14 to 24, as long as the effect of the present invention is not interfered with. The content of the lipid is generally not more than 40 mol %, preferably not more than 20 mol %, more preferably not more than 10 mol %, still more preferably not more than 5 mol % (for example, substantially 0 mol %).

The phospholipid membrane used in the present invention can be obtained by blending or processing constituents such as a phospholipid, a reactive phospholipid, a phospholipid membrane stabilizer, an antigen, and adding this mixture to an appropriate solvent or by other methods.

For example, when the phospholipid membrane is a liposome, production methods such as extrusion, the vortex mixer method, sonication, surfactant removal, reversed-phase evaporation, ethanol injection, the pre-vesicle method, the French press method, the W/O/W emulsion method, annealing, and freeze and fusion method can be mentioned. The form of the liposome is not subject to limitation; by choosing one of the aforementioned liposome production methods as appropriate, liposomes with various sizes and forms such as a multi-layer liposome, a small mono-layer membrane liposome, and a large mono-layer membrane liposome can be produced.

Although the particle diameter of the liposome is not subject to limitation, considering storage stability and the like, particle diameters of 20 to 600 nm can be mentioned and the particle diameter is preferably 30 to 500 nm, more preferably 40 to 400 nm, still more preferably 50 to 300 nm, most preferably 70 to 230 nm.

Also when the phospholipid membrane is a phospholipid miselle, the phospholipid membrane can be obtained by the same processes as those described above.

In the present invention, to improve the physicochemical stability of the liposome, a saccharide or a polyhydric alcohol may be added to the inner aqueous phase and/or outer aqueous phase of the liposome during or after preparation of the liposome. Particularly, if long-term storage or storage during the course of preparation making is required, it is preferable that a saccharide or a polyhydric alcohol be added and dissolved as a liposome protectant, and the water content be removed by freeze drying to obtain a freeze-dried product of a phospholipid composition.

As examples of the saccharide, monosaccharides such as glucose, galactose, mannose, fructose, inositol, ribose, and xylose; disaccharides such as saccharose, lactose, cellobiose, trehalose, and maltose; trisaccharides such as raffinose and melezitose; oligosaccharides such as cyclodextrin; polysaccharides such as dextrin; sugar alcohols such as xylitol, sorbitol, mannitol and maltitol, and the like can be mentioned. Of these saccharides, monosaccharides or disaccharides are preferable; in particular, glucose or saccharose is more preferably used because of their availability and the like.

As examples of the aforementioned polyhydric alcohol, glycerin compounds such as glycerin, diglycerin, triglycerin, tetraglycerin, pentaglycerin, hexaglycerin, heptaglycerin, octaglycerin, nonaglycerin, decaglycerin, and polyglycerin; sugar alcohol compounds such as sorbitol and mannitol; ethylene glycol, diethylene glycol, triethylene glycol, tetraethylene glycol, pentaethylene glycol, hexaethylene glycol, heptaethylene glycol, octaethylene glycol, nonaethylene glycol and the like can be mentioned. Of these alcohols, glycerin, diglycerin, triglycerin, sorbitol, mannitol, and polyethylene glycols having molecular weights of 400 to 10,000 are preferably mentioned because of their availability.

The concentration of the saccharide or polyhydric alcohol contained in the inner aqueous phase and/or outer aqueous phase of the liposome is, for example, 1 to 20% by weight, preferably 2 to 10% by weight based on concentration by weight relative to the liposome suspension.

For preparation of the T cell activator of the present invention, a phospholipid membrane prior to antigen binding is prepared, and then the antigen is bound to the membrane, whereby the T cell activator can be conveniently obtained.

For example, a phospholipid membrane, such as liposome suspension, comprising a phospholipid, a phospholipid membrane stabilizer and a reactive phospholipid for binding the antigen to the membrane surface, is prepared, and sucrose, one of the aforementioned saccharides, is added to, and dissolved in the outer aqueous phase at about 2 to 10% by weight. This saccharide-added preparation is transferred to a 10 ml glass vial, placed in a shelf-rack freeze-drier, and cooled to −40° C. and the like to freeze the sample, after which a freeze-dried product is obtained by a conventional method.

Since the freeze-dried product of the phospholipid membrane thus obtained is free of water content, a long-term preservation is possible; where necessary, a particular antigen is added and subjected to subsequent steps, whereby the final T cell activator of the present invention can be obtained conveniently and quickly. When the interaction between the antigen and the phospholipid membrane is strong and the instability is significant, it is very convenient that the phospholipid membrane is stored in the form of a freeze-dried product and used after being bound with the antigen when necessary, as described above.

The phospholipid membrane contained in the T cell activator of the present invention can have an antigen-bound phospholipid. As the method of obtaining a phospholipid membrane comprising an antigen-bound phospholipid, the following methods (A) and (B) can be mentioned:

(A) A phospholipid membrane comprising a phospholipid, a reactive lipid and a phospholipid membrane stabilizer is prepared, and an antigen and a bifunctional reactive compound are added thereto to join the functional group of the reactive phospholipid contained in the phospholipid membrane and the functional group of the antigen via the bifunctional reactive compound. The bifunctional reactive compound that can be used here may be the same as that used to prepare a terminally modified product of the reactive phospholipid. Specifically, as the bifunctional reactive compound comprising an aldehyde group, glyoxal, glutaraldehyde, succindialdehyde, terephthalaldehyde and the like can be mentioned. Preferably, glutaraldehyde can be mentioned. Furthermore, as the bifunctional reactive compound having a succinic acid imide group, dithiobis(succinimidylpropionate), ethylene glycol-bis(succinimidylsuccinate), disuccinimidylsuccinate, disuccinimidylsuberate, disuccinimidylglutarate and the like can be mentioned. As the bifunctional reactive compound having a succinimide group at one terminal and a maleimide group at the other terminal, N-succinimidyl-4-(p-maleimidephenyl)butyrate, sulfosuccinimidyl-4-(p-maleimidephenyl)butyrate, N-succinimidyl-4-(p-maleimidephenyl)acetate, N-succinimidyl-4-(p-maleimidephenyl)propionate, succinimidyl-4-(N-maleimidethyl)-cyclohexane-1-carboxylate, sulfosuccinimidyl-4-(N-maleimidethyl)-cyclohexane-1-carboxylate, N-(γ-maleimidebutyryloxy)succinimide, N-(ε-maleimidecaproyloxy)succinimide and the like can be used. Using such a bifunctional reactive compound, a terminally modified product of a reactive phospholipid (for example, phosphatidylethanolamine) having a maleimide group as the functional group is obtained.

(B) Method comprising preparing a phospholipid membrane comprising a phospholipid, a reactive phospholipid, and a phospholipid membrane stabilizer, adding an antigen thereto, and joining the functional group of the reactive phospholipid contained in the phospholipid membrane and the functional group of the antigen to bind the antigen.

As examples of the kinds of bonds in the aforementioned (A) and (B), an ionic bond, a hydrophobic bond, a covalent bond and the like can be mentioned, and a covalent bond is preferable. As specific examples of the covalent bond, a Schiff's base bond, an amide bond, a thioether bond, an ester bond and the like can be mentioned.

The two methods described above both enable the binding of an antigen to the reactive phospholipid contained in the phospholipid membrane, resulting in the formation of an antigen-bound phospholipid in the phospholipid membrane.

As a specific example of the method of binding the starting material phospholipid membrane and the antigen via a bifunctional reactive compound in the aforementioned method (A), a method utilizing a Schiff's base bond can be mentioned. As the method of binding the phospholipid membrane and the antigen via a Schiff's base bond, a method comprising preparing a phospholipid membrane having an amino group on the surface thereof, adding the antigen to a suspension of the phospholipid membrane, then adding dialdehyde as the bifunctional reactive compound, and binding the amino group on the phospholipid membrane surface and the amino group in the antigen via a Schiff's base, can be mentioned.

As specific examples of this binding procedure, the following methods can be mentioned.

(A-1) To obtain a phospholipid membrane having an amino group on the surface thereof, a reactive phospholipid having an acyl group having one unsaturated bond and 14 to 24 carbon atoms or a hydrocarbon group having one unsaturated bond and 14 to 24 carbon atoms (e.g., phosphatidylethanolamine) is mixed in a phospholipid membrane starting material lipid (phospholipid, phospholipid membrane stabilizer and the like) to prepare a phospholipid membrane wherein a given amount of amino groups is present on the phospholipid membrane surface.

(A-2) An antigen is added to the aforementioned phospholipid membrane suspension.

(A-3) Then, as the bifunctional reactive compound, glutaraldehyde is added, and mixture is allowed to react for a specified time to form Schiff's base bond between the phospholipid membrane and the antigen.

(A-4) Subsequently, to inactivate the reactivity of residual glutaraldehyde, glycine, as the amino group-containing water-soluble compound, is added to the phospholipid membrane suspension to allow the reaction.

(A-5) By methods such as gel filtration, dialysis, ultrafiltration, centrifugation and the like, the antigen not bound to the phospholipid membrane, the reaction product of glutaraldehyde and glycine, and excess glycine are removed to give a suspension of an antigen-bound phospholipid membrane.

As a specific example of the aforementioned method (B), a method comprising introducing a reactive phospholipid having a functional group capable of forming an amide bond, a thioether bond, a Schiff's base bond, an ester bond or the like into the phospholipid membrane can be mentioned. As specific examples of the functional group, a succinimide group, a maleimide group, an amino group, an imino group, a carboxyl group, a hydroxyl group, a thiol group and the like can be mentioned.

As an example of the reactive phospholipid to be introduced into the phospholipid membrane, an amino terminal modified product of the aforementioned reactive phospholipid having an acyl group having one unsaturated bond and 14 to 24 carbon atoms or a hydrocarbon group having one unsaturated bond and 14 to 24 carbon atoms (e.g., phosphatidylethanolamine) can be used.

Specific examples of this binding procedure are hereinafter described with reference to the case using diacylphosphatidylethanolamine.

(B-1) A diacylphosphatidylethanolamine having an acyl group having one unsaturated bond and 14 to 24 carbon atoms, and disuccinimidylsuccinate are reacted only at one terminal thereof by a known method to give a disuccinimidylsuccinate-bound diacylphosphatidylethanolamine having a succinimide group as the functional group on one terminal thereof.

(B-2) The aforementioned disuccinimidylsuccinate-bound diacylphosphatidylethanolamine and another phospholipid membrane constituent (phospholipid, phospholipid membrane stabilizer and the like) are mixed by a known method to give a phospholipid membrane composition having a succinimide group as the functional group on the surface thereof.

(B-3) An antigen is added to the aforementioned suspension of the phospholipid membrane composition to allow reaction of the amino group in the antigen with the succinimide group on the surface of the phospholipid membrane.

(B-4) The unreacted antigen, reaction byproducts and the like are removed by methods such as gel filtration, dialysis, ultrafiltration, and centrifugation to give a suspension of a phospholipid membrane comprising an antigen-bound phospholipid.

When a phospholipid membrane and an antigen are bound, because the antigen is mainly a protein or a peptide, an amino group or a thiol group frequently contained as a reactive group is preferably used as a target in practice. When an amino group is the target, a Schiff's base bond can be formed by reacting the amino group with a succinimide group. When a thiol group is the target, a thioether bond can be formed by reacting the thiol group with a maleimide group.

Using the T cell activator of the present invention, it is possible to potently activate cytotoxic T lymphocytes (CTL). It is known that cytotoxic T lymphocytes are major effector cells in cellular immunity, and kill cells infected with intracellular infectious pathogens (virus, malaria protozoa and the like) or tumor cells in an antigen-specific manner to remove these cells. Cytotoxic T lymphocytes are generally T cells having the CD8+ phenotype. Therefore, by administering the T cell activator of the present invention to patients with viral infectious diseases such as poliomyelitis, influenza, Japanese encephalitis, measles, rubella, mumps, rabies, yellow fever, varicella, hepatitis A, hepatitis B, hepatitis C, hemorrhagic fever with renal syndrome, dengue hemorrhagic fever, rotavirus infections, parvovirus, corona virus, distemper virus, leptospira, infectious bronchitis virus, contagious leukemia virus, AIDS, SARS, highly pathogenic avian influenza virus, and pediatric diarrhea virus; bacterial infectious diseases such as tuberculosis, pertussis, diphtheria, tetanus, and cholera; infectious diseases caused by intracellular infectious bacteria such as mycoplasma; infectious diseases caused by intracellular parasitic protozoas such as plasmodium and schistosomes; cancers (lung cancer, breast cancer, colorectal cancer, liver cancer, pancreas/gall bladder cancer, uterine cervical cancer, uterine body cancer, ovarian cancer, choriocarcinoma, prostatic cancer, gastric cancer, and the like) and the like, it is possible to activate cytotoxic T lymphocytes (CTL) in the patients, and to prevent/treat the diseases. Hence, the T cell activator of the present invention is useful as a prophylactic/therapeutic agent for the above-described diseases such as infectious diseases and cancers.

When the T cell activator of the present invention is used as the above-described prophylactic/therapeutic agent and the like, it can be prepared as a preparation according to a conventional method. The T cell activator of the present invention is of low toxicity, and can be administered orally or parenterally (e.g., intravascular administration, subcutaneous administration and the like) as a liquid as is, or as a pharmaceutical composition in an appropriate dosage form, to humans, non-human mammals (e.g., rats, rabbits, sheep, pigs, bovines, cats, dogs, monkeys and the like), birds (chicken, geese, domestic ducks, ostriches, partridges and the like), fishes (salmon, trout, Japanese amberjack, greater amberjack, young yellowtail, sea bream, flatfish, carp and the like) and the like. The T cell activator of the present invention is generally administered parenterally.

The T cell activator of the present invention permits administration of the antibody-bound phospholipid membrane, which is the active ingredient thereof, per se, or may be administered as an appropriate pharmaceutical composition. The pharmaceutical composition used for the administration may comprise the above-described antibody-bound phospholipid membrane, a pharmaceutically acceptable carrier, and a diluent or an excipient. Such a pharmaceutical composition is provided as a dosage form suitable for oral or parenteral administration.

As examples of the composition for parenteral administration, an injection, a suppository and the like are used; the injection may encompass dosage forms such as preparations for intravenous injection, subcutaneous injection, intradermal injection, intramuscular injection, and infusion. Such an injection can be prepared according to a commonly known method. Regarding the method of preparing the injection, it can be prepared by, for example, suspending the above-described antibody-bound phospholipid membrane in an aseptic aqueous solvent normally used for injection. As examples of the aqueous solvent for injection, distilled water; physiological saline; buffer solutions such as phosphate buffer solution, carbonate buffer solution, Tris-buffer solution, and acetate buffer solution, and the like can be used. The pH of such an aqueous solvent may be 5 to 10, preferably 6 to 8. The injection liquid prepared is preferably filled in an appropriate ampoule.

It is also possible to prepare a powder of the antibody-bound phospholipid membrane by subjecting a suspension of the antibody-bound phospholipid membrane to a process such as vacuum drying or freeze-drying. The antibody-bound phospholipid membrane may be preserved in a powder state, and the powder may be dispersed in an aqueous solvent for injection at the time of use.

The present invention is hereinafter described in more specifically by means of the following Examples, but this invention is not limited thereto.

### EXAMPLES

The compositions of the liposomes used in the Examples are shown in Table 1.

TABLE 1

| | carbon | number of | | | | | | | | | | |
|---|---|---|---|---|---|---|---|---|---|---|---|---|
| kind of | number of | double | Com. | Com. | Com. | Com. | Com. | | | | | |
| lipid | acyl group | bond | Ex. 1 | Ex. 2 | Ex. 3 | Ex. 4 | Ex. 5 | Ex. 1 | Ex. 2 | Ex. 3 | Ex. 4 | Ex. 5 |
| PC | 12 | 0 | — | 25.00 | — | — | — | — | — | — | — | — |
| | 14 | 0 | — | — | 25.00 | — | — | — | — | — | — | — |
| | 16 | 0 | — | — | — | 25.00 | — | — | — | — | — | — |
| | 18 | 0 | — | — | — | — | 37.5 | — | — | — | — | — |
| | 18 | 1 | — | — | — | — | — | 25.00 | 25.00 | 25.00 | 22.5 | 37.5 |
| PE | 12 | 0 | — | 18.75 | — | — | — | — | — | — | — | — |
| | 14 | 0 | — | — | 18.75 | — | — | — | — | — | — | — |
| | 16 | 0 | — | — | — | — | — | — | — | — | — | — |
| | 18 | 0 | — | — | — | 18.75 | — | — | — | — | — | — |
| | 18 | 1 | — | — | — | — | — | 18.75 | 18.75 | 13.75 | — | — |
| DSS-PE | 16 | 0 | — | — | — | — | 6.25 | — | — | — | — | — |
| EMCS-PE | 18 | 1 | — | — | — | — | — | — | — | — | 21.25 | — |
| | 18 | 1 | — | — | — | — | — | — | — | — | — | 6.25 |
| cholesterol | | | 0 | 43.75 | 43.75 | 43.75 | 43.75 | 43.75 | 43.75 | 36.25 | 43.75 | 43.75 |
| PG | 12 | 0 | — | 12.5 | — | — | — | — | — | — | — | — |
| | 14 | 0 | — | — | 12.5 | — | — | — | — | | — | — |
| | 16 | 0 | — | — | — | 12.5 | — | — | — | — | — | — |
| | 18 | 0 | — | — | — | — | 12.5 | — | — | — | — | — |
| | 18 | 1 | — | — | — | — | — | 12.5 | — | — | 12.5 | 12.5 |
| PS | 10 | 0 | — | — | — | — | — | — | — | — | — | — |
| | 12 | 0 | — | — | — | — | — | — | — | — | — | — |
| | 14 | 0 | — | — | — | — | — | — | — | — | — | — |
| | 16 | 0 | — | — | — | — | — | — | — | — | — | — |
| | 18 | 0 | — | — | — | — | — | — | — | — | — | — |
| | 18 | 1 | — | — | — | — | — | — | 12.5 | 25 | — | — |

PC: diacylphosphatidylcholine,
PS: diacylphosphatidylserine,
PG: diacylphosphatidylglycerol,
PE: diacylphosphatidylethanolamine,
DSS-PE: amino group-reactive phospholipid (succinimidyl group-PE),
EMCS-PE: thiol-reactive phospholipid (maleimide group-PE)

### Reference Example 1

### Preparation of Liposome 1

1) Preparation of Lipid-Mixed Powder

0.7560 g (1.2157 mmol) of dioleoylphosphatidylcholine, 0.5287 g (0.9118 mmol) of dioleoylphosphatidylethanolamine, 0.8225 g (2.1274 mmol) of cholesterol and 0.3927 g (0.6078 mmol) of dioleoylphosphatidylglycerol sodium salt were charged in an eggplant-shaped flask, 50 ml of a chloroform/methanol/water (65/25/4, ratio by volume) mixed solvent was placed therein to allow dissolution at 40° C. Next, the solvent was evaporated under reduced pressure using a rotary evaporator to form a thin layer of lipid. Furthermore, 30 ml of distilled water for injection was added, and the mixture was stirred to yield a homogenous slurry. This slurry was frozen with liquid nitrogen and dried in a freeze-drier for 24 hours to give a lipid-mixed powder.

2) Preparation of Liposome

Then, 60 ml of a separately prepared buffer solution (0.12 mM Na₂HPO₄, 0.88 mM KH₂PO₄, 0.25 M saccharose, pH 6.5, hereinafter to be abbreviated as “buffer solution”) was placed in an eggplant-shaped flask containing the above-described lipid-mixed powder, and the lipid was hydrated with stirring at 40° C. to give a liposome. Then, the particle diameter of the liposome was adjusted using an extruder. First, the obtained liposome was passed through an 8 μm polycarbonate filter, and subsequently passed through filters of 5 μm, 3 μm, 1 μm, 0.65 μm, 0.4 μm and 0.2 μm pore sizes in this order. Liposome particles having an average particle diameter of 191 nm (determined by the dynamic light scattering method) were obtained.

### Reference Example 2

### Preparation of Liposome 2

1) Synthesis of Reactive Phospholipid Consisting of Terminally Modified phosphatidylethanolamine (succinimidyl-dioleoyl phosphatidylethanolamine)

2 g of dioleoylphosphatidylethanolamine and 180 μl of triethylamine were added to, and dissolved in, 50 ml of chloroform, and the mixture was placed in a four-mouthed flask of 300 ml capacity. While this flask was being stirred at room temperature using a magnet stirrer, a separately prepared solution of 3 g of disuccinimidylsuccinate, which is a bifunctional reactive compound, dissolved in 80 ml of chloroform, was added dropwise by a conventional method for 4 hours, to allow one end of disuccinimidylsuccinate to react with the amino group of dioleoylphosphatidylethanolamine. This crude reaction mixture was transferred to an eggplant-shaped flask, and the solvent was evaporated using an evaporator. Next, a small amount of chloroform sufficient to dissolve the crude reaction product was added to this flask to yield a high-concentration crude reaction product solution, which was then subjected to column chromatography by a conventional method using silica gel equilibrated with chloroform/methanol/water (65/25/1, ratio by volume). Only the desired fraction wherein one terminal of disuccinimidylsuccinate is bound to an amino group of dioleoylphosphatidylethanolamine was recovered, and the solvent was evaporated, to yield succinimidyl-dioleoyl phosphatidylethanolamine, which is the object reactive phospholipid.

2) Preparation of Lipid-Mixed Powder

0.0337 g (0.0541 mmol) of dioleoylphosphatidylcholine, 0.2165 g (0.2705 mmol) of the succinimidyl-dioleoylphosphatidylethanolamine prepared in the previous term, 0.5021 g (1.2986 mmol) of cholesterol and 1.7477 g (2.706 mmol) of dioleoylphosphatidylglycerol sodium salt were charged in an eggplant-shaped flask, 50 ml of a chloroform/methanol/water (65/25/4, ratio by volume) mixed solvent was placed therein to allow dissolution at 40° C. Next, the solvent was evaporated under reduced pressure using a rotary evaporator to form a thin layer of lipid. Furthermore, 30 ml of distilled water for injection was added, and the mixture was stirred to yield a homogenous slurry. This slurry was frozen in liquid nitrogen and dried in a freeze-drier for 24 hours to give a lipid-mixed powder.

3) Preparation of Liposome

In the same manner as 2) Preparation of liposome in Reference Example 1 above, a liposome was prepared. Liposome particles having an average particle diameter of 224 nm (determined by the dynamic light scattering method) were obtained.

### Reference Example 3

### Preparation of Liposome 3

1) Synthesis of Reactive Phospholipid Consisting of Terminally Modified phosphatidylethanolamine (maleimide-dioleoyl phosphatidylethanolamine)

Using N-succinimidyl-4-(p-maleimidephenyl)propionate in place of disuccinimidylsuccinate in 1) Synthesis of reactive phospholipid consisting of terminally modified phosphatidylethanolamine in Reference Example 2 above, and using the same mol number of dioleoylphosphatidylethanolamine, triethylamine and a bifunctional reactive compound and similarly performing subsequent steps, maleimide-dioleoylphosphatidylethanolamine, which is the desired reactive phospholipid, was obtained.

2) Preparation of Lipid-Mixed Powder

1.0425 g (1.8428 mmol) of dioleoylphatidylcholine, 0.2375 g (0.3071 mmol) of the maleimide-dioleoyl phosphatidylethanolamine prepared in the previous term, 0.8313 g (2.1499 mmol) of cholesterol and 0.3888 g (0.6143 mmol) of dioleoylphosphatidylglycerol sodium salt were charged in an eggplant-shaped flask, 50 ml of a chloroform/methanol/water (65/25/4, ratio by volume) mixed solvent was placed therein to allow dissolution at 40° C. Next, the solvent was evaporated under reduced pressure using a rotary evaporator to form a thin layer of lipid. Furthermore, 30 ml of distilled water for injection was added, and the mixture was stirred to give a homogenous slurry. This slurry was frozen in liquid nitrogen and dried in a freeze-drier for 24 hours to give a lipid-mixed powder.

3) Preparation of Liposome

In the same manner as 2) Preparation of liposome in Reference Example 1 above, a liposome was prepared. Liposome particles having an average particle diameter of 186 nm (determined by the dynamic light scattering method) were obtained.

### Example 1

### Immunization Test by Administration of Ova-Bound Liposome Suspension

1) Preparation of Liposome Preparation;

2 ml of the liposome of Reference Example 1 was placed in a test tube, and 0.5 ml of a solution (12 mg/ml) of ovalbumin (manufactured by Sigma Company, reagent, hereinafter also referred to as OVA) was added. Next, 0.5 ml of 2.4% glutaraldehyde solution was added dropwise, the mixture was gently mixed on a 37° C. warm bath for 1 hour to immobilize the ovalbumin on the outer aqueous phase side of the liposome. Next, 0.5 ml of 2 M glycine-NaOH buffer solution (pH 7.2) was added, and the solution was allowed to stand at 4° C. overnight to inactivate the unreacted glutaraldehyde. Furthermore, this solution was passed through a column packed with Sepharose CL-4B (Pharmacia Biotech Company, trademark) to fractionate the object product, and to yield a liposome suspension wherein the antigen is bound to the surface thereof. The phosphorus concentration in the aforementioned liposome suspension was measured (Phospholipid Test Wako), and the phospholipid-derived phosphorus concentration was adjusted to 2 mM by dilution with the buffer solution to yield a suspension of OVA-bound liposome.

The same procedure as described above was performed using radiolabeled OVA separately, and the amount of OVA bound when the phosphorus concentration derived from phospholipid of the liposome was 2 mM was measured and found to be 49 μg/ml.

2) Methods of Measuring CD4+ T Cell Activity, CD8+ T Cell Activity and CTL Activity;

(Mice)

BALB/c mice (8 weeks of age, female) were purchased from Charles River Japan (Yokohama, Kanagawa, Japan). C57BL/6 mice (6-8 weeks of age, female) were purchased from SLC (Shizuoka, Japan). All mice were maintained under specific-pathogen-free (SPF) conditions.

(Preparation of Spleen Adherent Cells (SAC) and CD4+ and CD8+ T Cells)

A splenocyte suspension was prepared using RPMI-1640 containing 10% FCS. Cells (5×10⁷ cells) in 5 ml of the medium containing 10% FCS were sown to a 50 mm plastic dish for tissue culture (No. #3002; Becton Dickinson Labware, Franklin Lakes, N.J.), and incubated in a humidified atmosphere in the presence of 5% CO₂ at 37° C. for 2 hours. After cultivation, non-adherent cells were removed by gentle washing in a warm medium, and then adherent cells were recovered using a cell scraper. CD4+ and CD8+ T cells were purified from splencytes (SC) of a mouse immunized with OVA-alum by means of the magnetic cell sorter system MACS in accordance with the manufacturer's protocol using anti-CD4 and anti-CD8 antibody-coated microbeads (Miltenyi Biotec GmbH). T cells were suspended in RPMI-1640 containing 10% FCS at a cell density of 2×10⁶ ml.

(Measurement of IL-5 Production by CD4⁺ and CD8⁺ T Cells)

Spleen adherent cells (SAC) and CD4⁺ and CD8⁺ T cells were prepared from splenocytes of a BALB/c mouse immunized with OVA-alum by the method described above. An OVA-bound liposome was added to the SAC culture, and the culture was incubated for 2 hours. The final concentration of the OVA-bound liposome added to the macrophage culture was 500 μg lipid/ml, containing 24 μg OVA/ml. For control, OVA was added to the culture at a final concentration of 24 μg/ml. The SAC was washed three times in ice-cooled medium; 2×10⁵ cells, were co-cultured in a 48-well plate (No. #3047; Becton Dickinson Labware, Franklin Lakes, N.J.) with 5×10⁵ CD4⁺ or CD8⁺ T cells. As an index of the activation of CD4+ T cells and CD8+ T cells, IL-5 concentrations in the culture supernatant were measured. As a result of a preliminary test, the optimum cultivation period for IL-5 production by CD4⁺ and CD8⁺ T cells under the above-described conditions was 5 days. After cultivation in a CO₂ incubator for 5 days, the culture supernatant was recovered and assayed for IL-5. The IL-5 in the culture supernatant was measured using the Biotrak™ mouse ELISA system (Amersham International, Buckinghamshire, UK). All test samples were assayed in duplicate, and the standard error in each test was constantly under 5% of the mean value.

(In Vivo Cytotoxic Assay (Measurement of CTL Activity))

For measuring CTL activity, C57BL/6 mice were used. Splenocytes of a C57BL/6 mouse were labeled with 0.5 or 5 μM CFDA-SE (Sigma) at room temperature for 15 minutes, and twice washed. Next, cells with bright CFSE (M2) were pulse labeled with 0.5 μg/ml OVA₂₅₇₋₂₆₄ at 37° C. for 90 minutes. Cells with dark CFSE (M1), as controls, were pulse labeled with the unrelated NP₃₆₆₋₃₇₄ (ASNENMDAM) peptide at 37° C. for 90 minutes. The cells were mixed in a 1:1 ratio, and a total of 5×10⁶ cells were intravenously injected to mice having 100 μg of anti-IL-10 monoclonal antibody 2A5, 5 μg of CpG and each OVA-bound liposome injected thereto 1 to 2 weeks previously. Eight hours later, splenocytes recovered from each mouse were analyzed by flowcytometry. The extent of the reduction in the fluorescently labeled splenocytes of the fraction pulse labeled with OVA₂₅₇₋₂₆₄ was used as an index of CTL activity. If CTL has been induced to the mouse immunized with the OVA-bound liposome, only the fluorescently labeled splenocytes of the fraction pulse labeled with OVA₂₅₇₋₂₆₄ would disappear.

3) Method of Measuring Antibody Production (IgG and IgE);

Using BALB/c mice (female, 8 weeks of age, 6 animals/group), an OVA-bound liposome suspension was intraperitoneally administered using an injection syringe at 200 μl/dose; 4 weeks later, the same suspension was administered by the same method to achieve secondary immunization. From the start of the experiment to 6 weeks later, serum was collected weekly, and changes in antibody titer (IgG and IgE) were measured by an ELISA method. Table 2 shows each antibody titer in serum at 6 weeks after start of the experiment.

TABLE 2

| | Com. | Com. | Com. | Com. | Com. | | | | | |
|---|---|---|---|---|---|---|---|---|---|---|
| Parameter | Ex. 1 | Ex. 2 | Ex. 3 | Ex. 4 | Ex. 5 | Ex. 1 | Ex. 2 | Ex. 3 | Ex. 4 | Ex. 5 |
| CD4 T cell | 350 | 800 | 910 | 880 | 650 | 1060 | 970 | 960 | 890 | 845 |
| activity | | | | | | | | | | |
| (IL-5 | | | | | | | | | | |
| (pg/ml)) | | | | | | | | | | |
| CD8 T cell | ND | ND | ND | ND | ND | 95 | 99 | 102 | 97 | 113 |
| activity | | | | | | | | | | |
| (IL-5 | | | | | | | | | | |
| (pg/ml)) | | | | | | | | | | |
| CTL | 0 | 0 | 0 | 0 | 0 | 100 | 100 | 100 | 100 | 100 |
| activity | | | | | | | | | | |
| (% | | | | | | | | | | |
| reduction) | | | | | | | | | | |
| IgG | 1200 | 880 | 240 | 220 | 160 | 1100 | 1250 | 1680 | 1800 | 1360 |
| (μg/ml) | | | | | | | | | | |
| IgE | ND | ND | ND | ND | ND | ND | ND | ND | ND | ND |
| (pg/ml) | | | | | | | | | | |
| Overall | X | X | X | X | X | ⊙ | ⊙ | ⊙ | ⊙ | ⊙ |
| rating | | | | | | | | | | |

### Example 2 and Example 3

### Immunization Test by Administration of OVA-Bound Liposome Suspension

1) According to the blending molar ratios of phospholipid and cholesterol shown in Table 1, the same amount of liposome was prepared by the same method as those in Reference Example 1 above. Next, suspensions of the OVA-bound liposomes in Example 2 and Example 3 were prepared in the same manner as Example 1 above.

The particle diameters of the liposomes of Example 2 and Example 3 were 190 and 165 nm, respectively.

When the phospholipid-derived phosphorus concentrations in Example 2 and Example 3 were 2 mM, the amounts of OVA bound were 52 and 42 μg/ml, respectively.

2) For the OVA-bound liposomes in Example 2 and Example 3, the activity of each type of cell and antibody production were evaluated in the same manner as Example 1. The results are shown in Table 2.

### Example 4

### Immunization Test by Administration of Ova-Bound Liposome Suspension

1) 1.5 ml of the liposome of Reference Example 2 was charged in a test tube, and 3 ml of a separately prepared solution of ovalbumin (manufactured by Sigma Company, reagent, hereinafter also referred to as OVA) (1.25 mM, in buffer solution) was added, and the mixture was gently stirred at 5° C. for 48 hours to allow the reaction. This reaction mixture was subjected to gel filtration by a conventional method using SepharoseCL-4B equilibrated with the buffer solution. Because the liposome fraction is turbid in white, the subject fraction can easily be identified, but it may be identified using an UV detector and the like.

The phosphorus concentration in the liposome suspension obtained here was measured (Phospholipid Test Wako), and the phospholipid-derived phosphorus concentration was adjusted to 2 mM by dilution with the buffer solution to yield a suspension of OVA-bound liposome.

When the phospholipid-derived phosphorus concentration was 2 mM, the amount of OVA bound was 38 μg/ml. 2) For the liposome in Reference Example 2, in the same manner as Example 1, the activity and antibody production of each type of cell were evaluated. The results are shown in Table 2.

### Example 5

### Immunization Test by Administration of Ova-Bound Liposome Suspension

1) 1.5 ml of the liposome of Reference Example 3 was charged in a test tube, and 3 ml of a separately prepared ovalbumin (manufactured by Sigma Company, reagent, hereinafter also referred to as OVA) solution (1.25 mM, in buffer solution) was added, and the mixture was gently stirred at 5° C. for 48 hours to allow the reaction. This reaction mixture was subjected to gel filtration by a conventional method using SepharoseCL-4B equilibrated with the buffer solution. Because the liposome fraction is turbid in white, the object fraction can easily be identified, but it may be identified using an UV detector and the like.

The phosphorus concentration in the liposome suspension obtained here was measured (Phospholipid Test Wako), and the phospholipid-derived phosphorus concentration was adjusted to 2 mM with the buffer solution to yield a suspension of OVA-bound liposome.

When the phospholipid-derived phosphorus concentration was 2 mM, the amount of OVA bound was 40 μg/ml. 2) In the same manner as Example 1, the activity and antibody production of each type of cell were evaluated. The results are shown in Table 2.

### Comparative Example 1

### Immunization Test by Administration of Aluminum Hydroxide Gel Suspension

1) Preparation of Aluminum Hydroxide Gel Suspension

OVA was dissolved in a separately prepared buffer solution (1.2 mm Na₂HPO₄, 8.8 mM KH₂PO₄, pH 6.5) to obtain a concentration of 500 μg/ml, and 1 ml of this OVA solution was added to 9 ml of an aluminum hydroxide gel suspension (500 μg/ml) prepared according to a conventional method to yield an OVA-aluminum hydroxide gel suspension. The OVA concentration of this OVA-aluminum hydroxide gel suspension is 50 μg/ml.

2) Antibody Production Test;

Using the OVA-aluminum hydroxide gel suspension prepared in the previous term, in place of the liposome preparation of Example 1, the activity of each type of cell and antibody production were evaluated in the same manner as Example 1. The results are shown in Table 2.

### Comparative Examples 2 to 4

### Immunization Test by Administration of OVA-Bound Liposome Suspension

1) According to the blending molar ratios of phospholipid and cholesterol shown in Table 1, the same amount of liposome was prepared by the same method as those in Reference Example 1 above. Next, suspensions of the OVA-bound liposomes in Comparative Examples 2 to 4 were prepared in the same manner as Example 1 above.

The particle diameters of the liposomes of Comparative Examples 2 to 4 were 272, 251, and 248 nm, respectively.

When the phospholipid-derived phosphorus concentration in Comparative Examples 2 to 4 was 2 mM, the amounts of OVA bound were 47, 51, and 52 μg/ml, respectively.

2) For Comparative Examples 2 to 4, the activity of each type of cell and antibody production were evaluated in the same manner as Example 1. The results are shown in Table 2.

### Comparative Example 5

### Immunization Test by Administration of OVA-bound Liposome Suspension

1) According to the blending molar ratios of phospholipid and cholesterol shown in Table 1, the same amount of liposome was prepared by the same method as those in Reference Examples 2 and 3 above. Next, using this liposome, a suspension of OVA-bound liposome was obtained in the same manner as Example 4 above. The particle diameter of the liposome of Comparative Example 5 was 251 nm. When the phospholipid-derived phosphorus concentration in Comparative Example 5 was 2 mM, the amount of OVA bound was 41 μg/ml.

2) For Comparative Example 5, the activity of each type of cell and antibody production were evaluated in the same manner as Example 1. The results are shown in Table 2.

Comprehensive Evaluation

When a phospholipid having a saturated acyl group having 12 to 18 carbon atoms was used as the phospholipid contained in the liposome, no enhancement of CD8+ T cell activity and CTL activity was observed (Comparative Examples 2 to 5). In contrast, when a phospholipid having an acyl group having one unsaturated bond and 18 carbon atoms (oleoyl group) was used, CD8+ T cell activity and CTL activity were potently enhanced (Examples 1 to 5).

From the results shown above, it was suggested that the T cell activator of the present invention may practically sufficiently enhance CD8 cell activity and CTL cell activity, is effective in removal of pathogen-infected cells and malignantly transformed cells, and is effective in the amelioration/treatment for infectious disease and cancer.

1. A method of activating T cells in a mammal, comprising administering an effective amount of an antigen-bound phospholipid membrane to the mammal, wherein the phospholipid membrane comprises a phospholipid having an acyl group having one unsaturated bond and 14 to 24 carbon atoms or a hydrocarbon group having one unsaturated bond and 14 to 24 carbon atoms, and a phospholipid membrane stabilizer, and the antigen is bound to the surface of the phospholipid membrane.

2. The method of claim 1, wherein the phospholipid is a phospholipid having an acyl group having one unsaturated bond and 14 to 24 carbon atoms.

3. The method of claim 2, wherein the acyl group is an oleoyl group.

4. The method of claim 2, wherein the phospholipid is at least one selected from among diacylphosphatidylserine, diacylphosphatidylglycerol, diacylphosphatidic acid, diacylphosphatidylcholine, diacylphosphatidylethanolamine, succinimidyl-diacylphosphatidylethanolamine, and maleimide-diacylphosphatidylethanolamine.

5. The method of claim 1, wherein the phospholipid membrane stabilizer is cholesterol.

6. The method of claim 1, wherein the antigen is bound to the phospholipid having an acyl group having one unsaturated bond and 14 to 24 carbon atoms or a hydrocarbon group having one unsaturated bond and 14 to 24 carbon atoms, contained in the phospholipid membrane.

7. The method of claim 1, wherein the antigen is an antigen derived from an intracellular infectious pathogen or a tumor antigen.

8. The method of claim 1, wherein the phospholipid membrane is a liposome.

9. The method of claim 1, wherein the T cells are CD8+ T cells.

10. The method of claim 1, wherein the T cells are CTLs.

11. The method of claim 1, wherein the phospholipid membrane has the following composition:
(A) a phospholipid having an acyl group having one unsaturated bond and 14 to 24 carbon atoms or a hydrocarbon group having one unsaturated bond and 14 to 24 carbon atoms, 1 to 99.8 mol %;
(B) a phospholipid membrane stabilizer, 0.2 to 75 mol %.

12. A method of activating T cells in a mammal, comprising administering an effective amount of an antigen-bound phospholipid membrane having the following composition to the mammal:
(I) an acidic phospholipid having an acyl group having one unsaturated bond and 14 to 24 carbon atoms or a hydrocarbon group having one unsaturated bond and 14 to 24 carbon atoms, 1 to 85 mol %;
(II) a neutral phospholipid having an acyl group having one unsaturated bond and 14 to 24 carbon atoms or a hydrocarbon group having one unsaturated bond and 14 to 24 carbon atoms, 0.01 to 80 mol %;
(III) an antigen-bound phospholipid having an acyl group having one unsaturated bond and 14 to 24 carbon atoms or a hydrocarbon group having one unsaturated bond and 14 to 24 carbon atoms, 0.2 to 80 mol %;
(IV) a phospholipid membrane stabilizer, 0.2 to 75 mol %.
